(19) United States
(12) Patent Application Publication
(10) Pub. No.: US 2026/0102325 A1
(43) Pub. Date: Apr. 16, 2026

(54) Personal Care Compositions

(71) Applicant: Colgate-Palmolive Company, New York, NY (US)

(72) Inventors: Viktor DUBOVOY, Basking Ridge, NJ (US); Haroon A. CHAUDHRY, Monroe Township, NJ (US); Zilma Pereira MUNEESWARAN, Highland Park, NJ (US); Ruchir PARIKH, East Windsor, NJ (US); Rahul PATEL, Hillsborough, NJ (US); Xiang CHEN, Somerset, NJ (US); Zachary MODEL, Westfield, NJ (US); Christine BOYKE, Somerset, NJ (US)

(73) Assignee: Colgate-Palmolive Company, New York, NY (US)

(21) Appl. No.: 19/117,269

(22) PCT Filed: Dec. 8, 2023
(86) PCT No.: PCT/US2023/083077

§ 371 Date: Mar. 31, 2025

Related U.S. Application Data
(60) Provisional application No. 63/431,476, filed on Dec. 9, 2022.
(60) Provisional application No. 63/431,479, filed on Dec. 9, 2022.
(60) Provisional application No. 63/431,485, filed on Dec. 9, 2022.

(51) Int. Cl. A61K 8/46; A61K 8/39; A61K 8/73; A61Q 19/10

(52) U.S. Cl. CPC A61K 8/463; A61K 8/39; A61K 8/466; A61K 8/73; A61Q 19/10; A61K 2800/596

(57) ABSTRACT

Personal care composition and methods of use are disclosed herein. In accordance with an aspect of the invention, provided is a personal care composition comprising from about 30 to about 85 wt. % of a surfactant system including a sulfate based anionic surfactant, and a non-sulfate based anionic surfactant; xanthan gum; and optionally, a fatty ester, wherein the personal care composition has a weight ratio of sulfate based anionic surfactant to non-sulfate based anionic surfactant ranging from about 8:1 to about 1:1, and all weight percentages are based on the total weight of the personal care composition.

### CROSS-REFERENCE TO RELATED APPLICATIONS

This application claims the benefit of priority from U.S. Provisional Application Nos. 63/431,485; 63/431,479; and 63/431,476, filed Dec. 9, 2022, the contents of which are hereby incorporated by reference herein in their entireties.

### BACKGROUND

Liquid cleansing compositions, such as hand soaps, body wash, etc., such as those in a filled hand soap dispenser have a large concentration of water, which contributes to the overall weight of the product. The weight of the product and size of the dispenser or container adds to the manufacturing, packaging costs, and shipping costs. Since clean water is available readily in most of the world, and packaging can be re-used, it is wasteful to ship water in the liquid cleansers from one location to another and to use the original packages only once.

Liquid cleansing compositions have a viscosity that is generally thicker than other cleansers and as a result have a desirable feel (lubricity) to the user when applied to their skin. Typically, adding water to a concentrate results in a diluted, thinner, i.e., reduced viscosity, solution. It is a challenge to develop a concentrated cleansing composition in a delivery vehicle that is easily soluble in water, achieves a viscosity desirable for such cleansing compositions upon dilution with water, and further achieves desirable amounts of foam upon dilution with water.

Additionally, the main functional ingredients in cleansing compositions are surfactants. Surfactants interact with water, thereby allowing it to “wet” surfaces more efficiently. The surfactant-water combination is then able to surround specks of dirt and carry them away with rinsing. Agitation of the water solution, for example by rubbing hands together during washing or lathering also aids the process of removing dirt.

Conventional cleansing compositions such as hand soaps contain surfactants in various amounts. Anionic surfactants are typically included because they provide foaming to a composition. Nonionic surfactants may also be included to provide cleansing, solubilizing, and dispersing properties, and are usually less irritating than anionic surfactants. Nonionic surfactants, however, often exhibit less foaming ability and do not provide any enhancement to viscosity (e.g., oftentimes the composition is thinner and runnier with increased amounts of nonionic surfactants). In some cleansing applications, higher viscosity is desired for the product's handling or ease of application. In addition, higher viscosity personal care products are more aesthetically appealing to many consumers. Transparent cleansing compositions is generally appealing to consumers, as there may be a belief that application of such compositions will make their skin transparent and young-looking.

The development of cleansing compositions has been driven by a need for certain performance properties that consumers find desirable. For example, consumers seek cleansing compositions that foam and cleanse well, have a certain “thickness” (viscosity), and are mild to the skin. The cleansing compositions should also rinse away from the body with ease. Often, the addition of a particular component to a cleansing composition will enhance one desired property to the detriment of another desired property. For instance, it is often difficult to achieve cleansing compositions having high concentrations of surfactants that are also transparent or translucent. It is therefore difficult to achieve a perfect balance of desirable performance properties.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention are directed to personal care compositions and, particularly, personal care compositions having desirable foaming and rheological properties and optionally a high transparency. In accordance with an aspect of the invention, provided is a personal care composition that includes from about 30 to about 85 wt. % of a surfactant system comprising a sulfate based anionic surfactant, and a non-sulfate based anionic surfactant; xanthan gum; and optionally a fatty ester, wherein the personal care composition has a weight ratio of sulfate based anionic surfactant to non-sulfate based anionic surfactant ranging from about 8:1 to about 1:1, and all weight percentages are based on the total weight of the personal care composition.

In accordance with another aspect, provided is a personal care composition that includes a surfactant system comprising a sulfate based anionic surfactant, and a non-sulfate based anionic surfactant; from about 0.05 to about 0.5 wt. % of xanthan gum; and optionally a fatty ester, wherein all weight percentages are based on the total weight of the personal care composition.

In accordance with another aspect, provided is a diluted personal care composition comprising a sulfate based anionic surfactant, and a non-sulfate based anionic surfactant; from about 0.005 to about 0.05 wt. % of xanthan gum; and optionally a fatty ester, wherein the personal care composition has a weight ratio of the sulfate based anionic surfactant to the non-sulfate based anionic surfactant ranging from about 8:1 to about 1:1, and all weight percentages are based on the total weight of the personal care composition.

### BRIEF DESCRIPTION OF THE DRAWINGS

The features, and advantages of the invention will be apparent from the following more detailed description of certain embodiments of the invention and as illustrated in the accompanying drawings in which:

FIG. 1 is a bar graph of the foam characteristic of a non-limiting, example composition and eight additional compositions according to aspects of the invention; and

FIG. 2 is a bar graph of the foam characteristics of several non-limiting, example compositions and additional compositions according to aspects of the invention.

It should be understood that the various aspects are not limited to the compositions, arrangements, and instrumentality shown in the figures.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other apparatuses and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the personal care composition. The symbol “°” refers to a degree, such as a temperature degree or a degree of an angle. The symbols “h”, “min”, “mL”, “nm”, and “μm” refer to hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

When referring to chemical structures, and names, the symbols “C”, “H”, and “O” mean carbon, hydrogen, and oxygen, respectively. The symbols “-”, “=” and “≡” mean single bond, double bond, and triple bond, respectively.

“Volatile”, as used herein, means having a flash point of less than about 100° C. “Non-volatile”, as used herein, means having a flash point of greater than about 100° C.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the personal care compositions of the instant disclosure can be free or essentially free of all components and elements positively recited throughout the instant disclosure. In some instances, the personal care compositions of the present disclosure may be substantially free of non-incidental amounts of the ingredient(s) or compound(s) described herein. A non-incidental amount of an ingredient or compound is the amount of that ingredient or compound that is added into the personal care composition by itself. For example, a personal care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the personal care composition includes both components (or the composition includes more than two components that overlap), an overlapping compound does not represent more than one component. For example, certain compounds may be characterized as both a surfactant and an emulsifier. If a particular personal care composition includes both a surfactant and an emulsifier, a compound that may be characterized as both a surfactant and an emulsifier will serve only as either a surfactant or an emulsifier—not both.

For readability purposes, the chemical functional groups are in their adjective form; for each of the adjectives, the word “group” is assumed. For example, the adjective “alkyl” without a noun thereafter, should be read as “an alkyl group.”

Aspects of the invention are directed to personal care compositions and, particularly personal care compositions having superior foaming rheological properties. The inventors discovered that personal care compositions having a surfactant system comprising certain surfactants in particular amounts and/or ratios in conjunction with certain thickeners can surprisingly produce diluted and/or ready-to-use personal care compositions having superior foaming characteristics with desirable rheological properties. Without being limited to any specific theories, it is believed that certain embodiments having a surfactant system and thickener(s) in particular amounts have a synergistic effect in yielding superior foaming characteristics simultaneously with desirable rheological properties. Additionally, the inventors discovered that utilizing the particular surfactant systems and thickener(s) described herein with certain fatty esters surprisingly achieved a personal care composition having improved transparency and an increased stability.

In accordance with an aspect of the invention, provided is a personal care composition that includes from about 30 to about 85 wt. % of a surfactant system comprising a sulfate based anionic surfactant, and a non-sulfate based anionic surfactant; xanthan gum; and optionally a fatty ester, wherein the personal care composition has a weight ratio of sulfate based anionic surfactant to non-sulfate based anionic surfactant ranging from about 8:1 to about 1:1, and all weight percentages are based on the total weight of the personal care composition.

In accordance with another aspect, provided is a personal care composition that includes a surfactant system comprising a sulfate based anionic surfactant, and a non-sulfate based anionic surfactant; from about 0.05 to about 0.5 wt. % of xanthan gum; and optionally a fatty ester, wherein all weight percentages are based on the total weight of the personal care composition.

In accordance with another aspect, provided is a diluted personal care composition comprising a sulfate based anionic surfactant, and a non-sulfate based anionic surfactant; from about 0.005 to about 0.05 wt. % of xanthan gum; and optionally a fatty ester, wherein the personal care composition has a weight ratio of the sulfate based anionic surfactant to the non-sulfate based anionic surfactant ranging from about 8:1 to about 1:1, and all weight percentages are based on the total weight of the personal care composition.

The personal care compositions may be formulated to be translucent and/or transparent in a non-diluted form, in a diluted and/or ready-to-use form, or in both a non-diluted form and in a diluted and/or ready-to-use form. As used herein, the term “translucent” refers to the characteristic of a personal care composition to permit the passage of light but does not necessarily allow for detailed objects to be distinguished through a thickness of 1 cm of the personal care composition. The term “transparent” with respect to personal care compositions of the present disclosure indicates that the personal care composition has transmittance of at least 80% at a wavelength of 600 nm, for example measured using a Lambda 40 UV-visible spectrometer, at a concentration of 0.5% by weight in water. The personal care compositions may have, for example, a transmittance of at least 80%, at least 85%, at least 88%, at least 90%, or at least 95% at a wavelength of 600 nm, measured, for example, using a Lambda 40 UV-visible spectrometer.

The personal care compositions may be formulated to have a viscosity of about 100 to about 1,500 cP using a Brookfield DV2T viscometer with a spindle number 5 at 20 RPM and a temperature of 25° C. in a non-diluted form. In some instances, the personal care composition has a viscosity of from about 100 to about 1,500 cP, about 100 to about 1,300 cP, about 100 to about 1,100 cP, about 100 to about 900 cP, about 100 to about 700 cP, about 100 to about 600 cP, about 100 to about 500 cP; from about 250 to about 1,500 cP, about 250 to about 1,300 cP, about 250 to about 1,100 cP, about 250 to about 900 cP, about 250 to about 700 cP, about 250 to about 600 cP, about 250 to about 500 cP; from about 350 to about 1,500 cP, about 350 to about 1,300 cP, about 350 to about 1,100 cP, about 350 to about 900 cP, about 350 to about 700 cP, about 350 to about 600 cP, about 350 to about 500 cP; from about 450 to about 1,500 cP, about 450 to about 1,300 cP, about 450 to about 1,100 cP, about 450 to about 900 cP, about 450 to about 700 cP, about 450 to about 600 cP; from about 550 to about 1,500 cP, about 550 to about 1,300 cP, about 550 to about 1,100 cP, about 550 to about 900 cP, about 550 to about 700 cP, or any range or subrange thereof using a Brookfield DV2T viscometer with a spindle number 5 at 20 RPM and a temperature of 25° C.

The personal care compositions may have a viscosity when in a non-diluted and/or concentrated form that is different from the viscosity of the personal care composition in a diluted and/or ready-to-use form. For example, the viscosity of the personal care composition in the diluted form may be greater than the viscosity of the personal care composition prior to dilution in the concentrated form. In some embodiments, the viscosity of the personal care composition in a diluted form is from about 1,000 cP to about 9,000 cP using a Brookfield DV2T viscometer with a spindle number 5 at 20 RPM and a temperature of 25° C. For instance, when diluted, the personal care composition may have a viscosity of about 1,000 to about 9,000 cP, about 1,000 to about 8,000 cP, about 1,000 to about 7,000 cP, about 1,000 to about 6,000 cP, about 1,000 to about 5,500 cP, about 1,000 to about 5,000 cP, about 1,000 to about 4,700 cP, about 1,000 to about 4,400 cP, about 1,000 to about 4,100 cP; from about 2,000 to about 9,000 cP, about 2,000 to about 8,000 cP, about 2,000 to about 7,000 cP, about 2,000 to about 6,000 cP, about 2,000 to about 5,500 cP, about 2,000 to about 5,000 cP, about 2,000 to about 4,700 cP, about 2,000 to about 4,400 cP, about 2,000 to about 4,100 cP; from about 3,000 to about 9,000 cP, about 3,000 to about 8,000 cP, about 3,000 to about 7,000 cP, about 3,000 to about 6,000 cP, about 3.000 to about 5,500 cP, about 3,000 to about 5,000 cP, about 3,000 to about 4,700 cP, about 3,000 to about 4,400 cP, about 3,000 to about 4,100 cP; from about 3,500 to about 9,000 cP, about 3,500 to about 8,000 cP, about 3,500 to about 7,000 cP, about 3,500 to about 6,000 cP, about 3,500 to about 5,500 cP, about 3,500 to about 5,000 cP, about 3,500 to about 4,700 cP, about 3,500 to about 4,400 cP, about 3,500 to about 4,100 cP; from about 3,800 to about 9,000 cP, about 3,800 to about 8,000 cP, about 3,800 to about 7,000 cP, about 3.800 to about 6,000 cP, about 3,800 to about 5,500 cP, about 3,800 to about 5,000 cP, about 3,800 to about 4,700 cP, about 3,800 to about 4,400 cP, about 3,800 to about 4,100 cP; from about 4,100 to about 9,000 cP, about 4,100 to about 8,000 cP, about 4, 100 to about 7,000 cP, about 4,100 to about 6,000 cP, about 4,100 to about 5,500 cP, about 4,100 to about 5,000 cP, about 4,100 to about 4,700 cP, about 4,100 to about 4,400 cP, or any range or subrange thereof, using a Brookfield DV2T viscometer with a spindle number 5 at 20 RPM and a temperature of 25° C.

The personal care compositions preferably have less than about 1 ppm of 1,4 dioxane. In some embodiments, the personal care compositions have 1 ppm or less, about 0.7 ppm or less, about 0.5 ppm or less, about 0.3 ppm or less, or about 0.1 ppm or less of 1,4 dioxane.

Suitable components, such as those listed below, may be included or excluded from the formulations for the personal care compositions depending on the specific combination of other components, the form of the personal care compositions, and/or the use of the formulation (e.g., a diluted/ready-to-use personal care composition, a cleansing composition, an antibacterial soap, etc.). According to certain aspects of the invention, the personal care composition is in the form of a concentrated personal care composition encapsulated in a pod. In some embodiments, the personal care composition is a cleansing composition in the form of a hand soap and/or a body wash.

The personal care compositions may include a surfactant system in an amount that may vary, but generally is in the range of from about 30 to about 85 wt. %, based on the total weight of the personal care composition. For example, the personal care composition may include a surfactant system in an amount from about 30 to about 80 wt. %, about 30 to about 75 wt. %, about 30 to about 70 wt. %, about 30 to about 65 wt. %, about 30 to about 60 wt. %, about 30 to about 55 wt. %; from about 40 to about 85 wt. %, about 40 to about 80 wt. %, about 40 to about 75 wt. %, about 40 to about 70 wt. %, about 40 to about 65 wt. %, about 40 to about 60 wt. %, about 40 to about 55 wt. %; from about 48 to about 85 wt. %, about 48 to about 80 wt. %, about 48 to about 75 wt. %, about 48 to about 70 wt. %, about 48 to about 65 wt. %, about 48 to about 60 wt. %, about 48 to about 55 wt. %; from about 52 to about 85 wt. %, about 52 to about 80 wt. %, about 52 to about 75 wt. %, about 52 to about 70 wt. %, about 52 to about 65 wt. %, about 52 to about 60 wt. %; from about 56 to about 85 wt. %, about 56 to about 80 wt. %, about 56 to about 75 wt. %, about 56 to about 70 wt. %, about 56 to about 65 wt. %; from about 60 to about 85 wt. %, about 60 to about 80 wt. %, about 60 to about 75 wt. %, about 60 to about 70 wt. %, about 60 to about 65 wt. %; from about 66 to about 85 wt. %, about 66 to about 80 wt. %, about 66 to about 75 wt. %, about 66 to about 70 wt. %, or any range or subrange thereof, based on the total weight of the personal care composition.

The surfactant systems disclosed herein typically comprise two or more surfactants selected from anionic surfactants, nonionic surfactants, amphoteric surfactants, zwitterionic surfactants, and combinations of two or more thereof. For example, the surfactant system may include two, three, four, five, six, seven, eight, or nine or more surfactants. In some embodiments, the surfactant system comprises from two to eight, from two to seven, from two to six, from two to five, from two to four, from three to seven, from three to six, from three to five, from four to eight, four to seven, four to six, or any range or subrange thereof. Preferably, the surfactant system may include two or more surfactants, with the two or more surfactants being chosen from anionic surfactants. In some embodiments, the surfactant system may include two or more surfactants chosen from different categories of anionic surfactants, nonionic surfactants, amphoteric surfactants, and zwitterionic surfactants. In some embodiments, however, the surfactant system of the personal care composition may consist of anionic surfactants.

The surfactant system of the personal care composition typically includes one or more sulfate based anionic surfactant(s) and one or more non-sulfate based anionic surfactants. The amount of sulfate based anionic surfactants may be from about 20 to about 70 wt. %, based on the total weight of the personal care composition. For example, the amount of sulfate based anionic surfactant(s) present in the personal care composition may be from about 20 to about 65 wt. %, about 20 to about 60 wt. %, about 20 to about 57 wt. %, about 20 to about 54 wt. %, about 20 to about 51 wt. %, about 20 to about 47 wt. %; from about 25 to about 70 wt. %, about 25 to about 65 wt. %, about 25 to about 60 wt. %, about 25 to about 57 wt. %, about 25 to about 54 wt. %, about 25 to about 51 wt. %, about 25 to about 47 wt. %; from about 30 to about 70 wt. %, about 30 to about 65 wt. %, about 30 to about 60 wt. %, about 30 to about 57 wt. %, about 30 to about 54 wt. %, about 30 to about 51 wt. %, about 30 to about 47 wt. %; from about 35 to about 70 wt. %, about 35 to about 65 wt. %, about 35 to about 60 wt. %, about 35 to about 57 wt. %, about 35 to about 54 wt. %, about 35 to about 51 wt. %, about 35 to about 47 wt. %; from about 40 to about 70 wt. %, about 40 to about 65 wt. %, about 40 to about 60 wt. %, about 40 to about 57 wt. %, about 40 to about 54 wt. %, about 40 to about 51 wt. %, about 40 to about 47 wt. %; from about 42.5 to about 70 wt. %, about 42.5 to about 65 wt. %, about 42.5 to about 60 wt. %, about 42.5 to about 57 wt. %, about 42.5 to about 54 wt. %, about 42.5 to about 51 wt. %; from about 45 to about 70 wt. %, about 45 to about 65 wt. %, about 45 to about 60 wt. %, about 45 to about 57 wt. %, about 45 to about 54 wt. %, about 45 to about 51 wt. %; from about 47.5 to about 70 wt. %, about 47.5 to about 65 wt. %, about 47.5 to about 60 wt. %, about 47.5 to about 57 wt. %, about 47.5 to about 54 wt. %; from about 50 to about 70 wt. %, about 50 to about 65 wt. %, about 50 to about 60 wt. %, about 50 to about 57 wt. %, or any range or subrange thereof, based on the total weight of the personal care composition.

The non-sulfate based anionic surfactant(s) may be present in the personal care composition in an amount from about 5 to about 30 wt. %, based on the total weight of the personal care composition. In some embodiments, the non-sulfate based anionic surfactant(s) is present in the personal care composition in an amount from about 5 to about 25 wt. %, about 5 to about 20 wt. %, about 5 to about 17 wt. %, about 5 to about 15 wt. %, about 5 to about 13 wt. %, about 5 to about 11 wt. %, about 5 to about 9 wt. %; from about 7 to about 30 wt. %, about 7 to about 25 wt. %, about 7 to about 20 wt. %, about 7 to about 17 wt. %, about 7 to about 15 wt. %, about 7 to about 13 wt. %, about 7 to about 11 wt. %; from about 10 to about 30 wt. %, about 10 to about 25 wt. %, about 10 to about 20 wt. %, about 10 to about 17 wt. %, about 10 to about 15 wt. %; from about 13 to about 30 wt. %, about 13 to about 25 wt. %, about 13 to about 20 wt. %, about 13 to about 17 wt. %; from about 16 to about 30 wt. %, about 16 to about 25 wt. %, about 16 to about 20 wt. %; from about 19 to about 30 wt. %, about 19 to about 25 wt. %, or any range or subrange thereof, based on the total weight of the personal care composition.

The surfactant system may comprise an anionic surfactant selected from sulfate anionic surfactants and/or salters thereof, including those selected from alkyl sulfates and alkyl ether sulfates. The alkyl sulfates and/or alkyl ether sulfates may have an alkyl group comprising 8 to 20 carbon atoms, 8 to 16 carbon atoms, 10 to 16 carbon atoms, or 10 to 13 carbon atoms. Non-limiting examples of alkyl sulfate and alkyl ether sulfate salts include sodium lauryl sulfate (SLS), sodium dodecyl sulfate (SDS), sodium lauryl ether sulfate (SLES), ammonium lauryl sulfate, and ammonium laureth sulfate. In some embodiments, the sulfate anionic surfactants include sodium lauryl sulfate, sodium laureth sulfate, a salt thereof, or a combination of two or more thereof.

The surfactant system may comprise one or more non-sulfate based surfactant, such as those categorized as acyl isethionates, acyl amino acids, alkyl sulfonates, alkyl sulfosuccinates, alkyl sulfoacetates, alkoxylated monoacids, salts thereof, or combinations of two or more thereof. The non-sulfate based surfactant may be selected from alkyl sulfonates, such as those having an alkyl group comprising 8 to 32 carbons. In some embodiments, the alkyl group of the alkyl sulfonates comprises from 8 to 28 carbons, 8 to 24 carbons, 8 to 20 carbons, 8 to 18 carbons, 8 to 16 carbons, 8 to 14 carbons; from 10 to 32 carbons, 10 to 28 carbons, 10 to 24 carbons, 10 to 20 carbons, 10 to 18 carbons, 10 to 16 carbons, 10 to 14 carbons; from 12 to 32 carbons, 12 to 28 carbons. 12 to 24 carbons, 12 to 20 carbons, 12 to 18 carbons, 12 to 16 carbons; from 14 to 32 carbons, 14 to 28 carbons, 14 to 24 carbons, 14 to 20 carbons, 14 to 18 carbons, 14 to 16 carbons, or ranges or subranges thereof. In some embodiments, the non-sulfate based surfactant comprises alpha olefin sulfonate having an alkyl group of 10 to 20 carbons, 12 to 18 carbons, or 14 to 16 carbons. Examples of sulfonate surfactants include alkyl aryl sulfonates (such as, sodium dodecyl benzene sulfonate, sodium dodecylbenzene sulfonate, and triethanolamine dodecylbenzene sulfonate), higher fatty acid esters of 1,2-dihydroxy propane sulfonate, sodium cocoyl monoglyceride sulfonate, a salt thereof, or a combination of two or more thereof.

Non-limiting examples of useful acyl isethionates include those having a structure according to the following formulas:

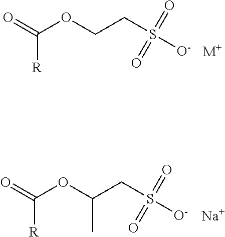

wherein R, R¹, R² and R³ are each independently selected from H or an alkyl chain having 1-24 carbons, with said chain being saturated or unsaturated, linear or branched, and X is COO or SO₃⁻. Sodium is shown as the cation in the above formula, but the cation may be an alkali metal ion such as sodium or potassium, ammonium ions, or alkanolammonium ions, such as monoethanolammonium or triethanolammonium ions. Non-limiting examples of acyl isethionates include sodium isethionate, sodium cocoyl isethionate, sodium lauroyl methyl isethionate, and sodium cocoyl methyl isethionate.

The non-sulfate anionic surfactants may be chosen from acyl amino acids. The acyl amino acids may include, but are not limited to, amino acid surfactants based on alanine, arginine, aspartic acid, glutamic acid, glycine, isoleucine, leucine, lysine, phenylalanine, serine, tyrosine, valine, sarcosine, threonine, and taurine. The cation associated with the acyl amino acid can be sodium or potassium. Alternatively, the cation can be an organic salt such as triethanolamine (TEA) or a metal salt.

Non-limiting examples of acyl sarcosinates include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, ammonium lauroyl sarcosinate, and a combination of two or more thereof. Examples of acyl taurates include those of the following formula:

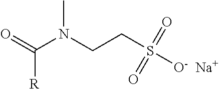

wherein R, R¹, R² and R³ are each independently selected from H or an alkyl chain having 1 to 24 carbon atoms, or from 6 to 20 carbon atoms, or from 8 to 16 carbon atoms, said chain being saturated or unsaturated, linear or branched, and X is COO⁻ or SO₃⁻. Non-limiting examples of acyl taurate salts include sodium cocoyl taurate, sodium methyl cocoyl taurate, oleoyl taurate, and combinations thereof.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, or a combination of two or more thereof.

Non-limiting examples of alkyl sulfacetates include, e.g., alkyl sulfoacetates such as C₄-C₁₈ fatty alcohol sulfoacetates and/or salts thereof. Useful cations for the salts include alkali metal ions, such as sodium or potassium, ammonium ions, or alkanolammonium ions, such as monoethanolammonium or triethanolammonium ions.

Examples of alkoxylated monoacids include, but are not limited to: butoxynol-5 carboxylic acid, butoxynol-19 carboxylic acid, capryleth-4 carboxylic acid, capryleth-6 carboxylic acid, capryleth-9 carboxylic acid, ceteareth-25 carboxylic acid, coceth-7 carboxylic acid, C₉₋₁₁ pareth-6 carboxylic acid, C₁₁₋₁₅ pareth-7 carboxylic acid, C₁₂₋₁₃ pareth-5 carboxylic acid, C₁₂₋₁₃ pareth-8 carboxylic acid, C₁₂₋₁₃ pareth-12 carboxylic acid, C₁₂₋₁₅ pareth-7 carboxylic acid, C₁₂₋₁₅ pareth-8 carboxylic acid, C₁₄₋₁₅ pareth-8 carboxylic acid, deceth-7 carboxylic acid, laureth-3 carboxylic acid, laureth-4 carboxylic acid, laureth-5 carboxylic acid, laureth-6 carboxylic acid, laureth-8 carboxylic acid, laureth-10 carboxylic acid, laureth-11 carboxylic acid, laureth-12 carboxylic acid, laureth-13 carboxylic acid, laureth-14 carboxylic acid, laureth-17 carboxylic acid, PPG-6-laureth-6 carboxylic acid, ppg-8-steareth-7 carboxylic acid, myreth-3 carboxylic acid, myreth-5 carboxylic acid, nonoxynol-5 carboxylic acid, nonoxynol-8 carboxylic acid, nonoxynol-10 carboxylic acid, octeth-3 carboxylic acid, octoxynol-20 carboxylic acid, oleth-3 carboxylic acid, oleth-6 carboxylic acid, oleth-10 carboxylic acid, ppg-3-deceth-2 carboxylic acid, capryleth-2 carboxylic acid, ceteth-13 carboxylic acid, deceth-2 carboxylic acid, hexeth-4 carboxylic acid, isosteareth-6 carboxylic acid, isosteareth-11 carboxylic acid, trudeceth-3 carboxylic acid, trideceth-6 carboxylic acid, trideceth-8 carboxylic acid, trideceth-12 carboxylic acid, trideceth-3 carboxylic acid, trideceth-4 carboxylic acid, trideceth-7 carboxylic acid, trideceth-15 carboxylic acid, trideceth-19 carboxylic acid, undeceth-5 carboxylic acid and/or a combination of two or more thereof.

The surfactant system may, in some embodiments, include one or more amphoteric surfactant(s). The amount of amphoteric surfactant(s) in the personal care composition may be from about 0.1 to about 15 wt. %, based on the total weight of the personal care composition. For instance, the amount of amphoteric surfactant in the personal care composition, if present, may be from about 0.1 to about 12 wt. %, about 0.1 to about 9 wt. %, about 0.1 to about 7 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 15 wt. %, about 0.5 to about 12 wt. %, about 0.5 to about 9 wt. %, about 0.5 to about 7 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 15 wt. %, about 1 to about 12 wt. %, about 1 to about 9 wt. %, about 1 to about 7 wt. %, about 1 to about 5 wt. %, about 1 to about 4 wt. %, about 1 to about 3 wt. %, about 1 to about 2 wt. %; from about 2 to about 15 wt. %, about 2 to about 12 wt. %, about 2 to about 9 wt. %, about 2 to about 7 wt. %, about 2 to about 5 wt. %, about 2 to about 4 wt. %, about 2 to about 3 wt. %; from about 3 to about 15 wt. %, about 3 to about 9 wt. %, about 3 to about 7 wt. %, about 3 to about 5 wt. %, about 3 to about 4 wt. %; from about 5 to about 15 wt. %, about 5 to about 12 wt. %, about 5 to about 9 wt. %, about 5 to about 7 wt. %, or any range or subrange thereof, based on the total weight of the personal care composition.

The amphoteric surfactant may comprise a substituent containing 8 to 18 carbon atoms and a substituent containing one or more carboxylate, sulfonate, sulfate, phosphate, or phosphonate. For instance, the amphoteric surfactant may have an alkyl group comprising 8 to 20 carbon atoms, 8 to 16 carbon atoms, 10 to 16 carbon atoms, or 10 to 13 carbon atoms. The amphoteric surfactant(s) may include, but are not limited to, derivatives of aliphatic secondary and tertiary amines in which the aliphatic radical can be straight chain or branched and wherein one of the aliphatic substituents contains about 8 to about 18 carbon atoms and one contains an anionic water solubilizing group, e.g., carboxy, sulfonate, sulfate, phosphate, or phosphonate.

The amphoteric surfactants may include alkyl amphoproprionates, betaines, alkyl sultaines, alkyl amphoacetates, and mixtures thereof. In some instances, the personal care compositions preferably include one or more alkyl amphopropionates. Non-limiting examples of alkyl amphopropionates include cocoamphopropionate, cornamphopropionate, caprylamphopropionate, cornamphopropionate, caproamphopropionate, oleoamphopropionate, isostearoamphopropionate, stearoamphopropionate, lauroamphopropionate, salts thereof, and a mixture thereof.

The personal care composition may include an amphoteric surfactant selected from betaine surfactants (also referred to as betaines). Examples of amphoteric surfactants include, but are not limited to, a range of betaines including, for example, high alkyl betaines, such as coco dimethyl carboxymethyl betaine, lauryl dimethyl carboxy-methyl betaine, lauryl dimethyl alpha-carboxyethyl betaine, cetyl dimethyl carboxymethyl betaine, lauryl bis-(2-hydroxyethyl) carboxy methyl betaine, stearyl bis-(2-hydroxypropyl) carboxymethyl betaine, oleyl dimethyl gamma-carboxypropyl betaine, and lauryl bis-(2-hydroxypropyl) alpha-carboxyethyl betaine, sulfobetaines such as coco dimethyl sulfopropyl betaine, stearyl dimethyl sulfopropyl betaine, amido betaines, amidosulfobetaines and the like. Preferably, a betaine surfactant is selected from coca betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, and combinations of two or more thereof. In some embodiments, at least one betaine surfactant is selected from coco betaine, cocamidopropyl betaine, behenyl betaine, capryl/capramidopropyl betaine, and lauryl betaine, and a combination of two or more thereof. In further embodiments, the betaine surfactant is selected from cocamidopropyl betaine, lauryl dimethyl betaine, cetyl dimethyl betaine, and cocoamphodiacetate, and a combination of two or more thereof. In at least one embodiment, the personal care composition comprises coco betaine, cocamidopropyl betaine, or a combination thereof.

The amphoteric surfactant may, in some cases, be selected sultaines, also known as sulfobetaines, and hydroxy sultaines, for example, cocamidopropyl hydroxysultain. The sultaines may be an alkyl sultaines having from 6 to 20 carbons, 8 to 18 carbons, 8 to 16 carbons, or 10 to 16 carbons. Examples of alkyl sultaine surfacants include, but are not limited to, cocamidopropyl hydroxysultaine, lauryl hydroxysultaine, and a combination of two or more thereof.

In some embodiments, the surfactant system includes an amphoteric surfactant selected from betaine surfactants and, optionally, an additional amphoteric surfactant other than betaines including those discussed above. The one or more betaine surfactants may, preferably, be selected from coca betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, and a combination of two or more thereof

The surfactant system may, in some embodiments, include one or more nonionic surfactant(s). The amount of nonionic surfactant in the personal care composition may be from about 0.1 to about 15 wt. %, based on the total weight of the personal care composition. For instance, the amount of nonionic surfactant in the personal care composition, if present, may be from about 0.1 to about 12 wt. %, about 0.1 to about 9 wt. %, about 0.1 to about 7 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 15 wt. %, about 0.5 to about 12 wt. %, about 0.5 to about 9 wt. %, about 0.5 to about 7 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 15 wt. %, about 1 to about 12 wt. %, about 1 to about 9 wt. %, about 1 to about 7 wt. %, about 1 to about 5 wt. %, about 1 to about 4 wt. %, about 1 to about 3 wt. %, about 1 to about 2 wt. %; from about 2 to about 15 wt. %, about 2 to about 12 wt. %, about 2 to about 9 wt. %, about 2 to about 7 wt. %, about 2 to about 5 wt. %, about 2 to about 4 wt. %, about 2 to about 3 wt. %; from about 3 to about 15 wt. %, about 3 to about 9 wt. %, about 3 to about 7 wt. %, about 3 to about 5 wt. %, about 3 to about 4 wt. %; from about 5 to about 15 wt. %, about 5 to about 12 wt. %, about 5 to about 9 wt. %, about 5 to about 7 wt. %, or any range or subrange thereof, based on the total weight of the personal care composition.

The nonionic surfactant may be selected from alkyl polyglucosides, alkylamides, alkanolamides, polyoxyalkylenated nonionic surfactants, polyglycerolated nonionic surfactants, ethoxylated fatty esters (preferably, polyglyceryl-2-caprate), and a combination of two or more thereof. In some cases, the nonionic surfactant may be selected from alkyl polyglucosides, alkylamides, and/or alkanolamides and may have an alkyl group having 8 to 32 carbons, 8 to 26 carbons, 8 to 22 carbons, 8 to 18 carbons, 10 to 22 carbons, 10 to 18 carbons, 12 to 22 carbons, or 12 to 18 carbons. In at least one preferred embodiment, the nonionic surfactant comprises an alkyl polyglucoside, an alkylamide, an alkanolamide, or a combination of two or more thereof having an alkyl group of 12 to 18 carbons.

Examples of alkanolamides include fatty acid alkanolamides. The fatty acid alkanolamides may be fatty acid monoalkanolamides, fatty acid dialkanolamides, fatty acid isoalkanolamides, or a mixture thereof, which may have a C₂₋₈ hydroxyalkyl group (the C₂₋₈ chain can be substituted with one or more than one OH group). Preferably, the fatty acid alkanolamides are chosen from fatty acid diethanolamides (DEA), fatty acid monoethanolamides (MEA), fatty acid monoisopropanolamides (MIPA), fatty acid diisopropanolamides (DIPA), fatty acid glucamides (acyl glucamides), or a combination of two or more thereof. In some preferred embodiments, the nonionic surfactant comprises cocamide monoethanolamine (MEA), cocamide diethanolamine (DEA), cocamide diisopropanolamine (DIPA), or a combination of two or more thereof. In at least one embodiment, the nonionic surfactant comprises cocamide DIPA.

Additional examples of fatty acid alkanolamides include oleic acid diethanolamide, myristic acid monoethanolamide, soya fatty acids diethanolamide, stearic acid ethanolamide, oleic acid monoisopropanolamide, linoleic acid diethanolamide, stearic acid monoethanolamide (Stearamide MEA), behenic acid monoethanolamide, isostearic acid monoisopropanolamide (isostearamide MIPA), erucic acid diethanolamide, ricinoleic acid monoethanolamide, coconut fatty acid monoisopropanolamide (cocoamide MIPA), coconut acid monoethanolamide (Cocamide MEA), palm kernel fatty acid diethanolamide, coconut fatty acid diethanolamide, lauric diethanolamide, polyoxyethylene coconut fatty acid monoethanolamide, coconut fatty acid monoethanolamide, lauric monoethanolamide, lauric acid monoisopropanolamide (lauramide MIPA), myristic acid monoisopropanolamide (Myristamide MIPA), coconut fatty acid diisopropanolamine (cocamide DIPA), or a combination of two or more thereof.

The nonionic surfactant may be selected from alkyl polyglucosides in some cases. In some instances, the alkyl polyglucoside(s) are chosen from lauryl glucoside, octyl glucoside, decyl glucoside, coco glucoside, caprylyl/capryl glucoside, sodium lauryl glucose carboxylate, and a combination of two or more thereof. In at least one embodiment, the personal care composition includes one or more nonionic surfactants comprising caprylyl glucoside and/or caprylyl/capryl glucoside. Non-limiting examples of nonionic surfactants worth mentioning include esters of polyols with fatty acids having a saturated or unsaturated chain, e.g., containing from 8 to 24 carbon atoms or 12 to 22 carbon atoms, and/or alkoxylated derivatives thereof. For example, the personal care composition may comprise polyethylene glycol esters of a C₈-C₂₄, such as C₁₂-C₂₂ glycol esters; sorbitol esters of a C₈-C₂₄, such as C₁₂-C₂₂ sorbitol esters; sugar (sucrose, glucose, alkylglycose) esters of a C₈-C₂₄; ethers of sugar and a C₈-C₂₄; or combinations of two or more thereof.

The nonionic surfactants may be selected from ethoxylated fatty esters, such as adducts of ethylene oxide with esters of lauric acid, palmitic acid, stearic acid, behenic acid, and mixtures thereof, including those containing from 9 to 100 oxyethylene groups. In some cases, the ethoxylated fatty esters may comprise PEG-9 laurate to PEG-50 laurate; PEG-9 palmitate to PEG-50 palmitate; PEG-9 stearate to PEG-50 stearate; PEG-9 palmitostearate to PEG-50 palmitostearate; PEG-9 behenate to PEG-50 behenate; polyethylene glycol 100 EO monostearate (such as, PEG-100 stearate); or combinations of two or more thereof. As examples of glyceryl esters of fatty acids, glyceryl stearate (glyceryl mono-, di- and/or tristearate), glyceryl ricinoleate, and a combination of two or more thereof may be incorporated in the personal care composition. Examples of glyceryl esters of C₈-C₂₄ alkoxylated fatty acids include polyethoxylated glyceryl stearate (glyceryl mono-, di- and/or tristearate), such as PEG-20 glyceryl stearate.

The personal care composition may, in some instances, include one or more cationic surfactant(s). For example, the personal care composition may include from about 0.1 wt. %, from about 0.5 wt. %, from about 1 wt. %, from about 2 wt. %, from about 3 wt. %, or from about 3.5 wt. % up to about 1 wt. %, up to about 2 wt. %, up to about 3 wt. %, up to about 4 wt. %, or up to about 5 wt. %, including any range or subrange formed therefrom, of cationic surfactant, based on the total weight of the personal care composition. Although some embodiment may include cationic surfactants, the personal care compositions may have cationic surfactants in an amount of about 5 wt. % or less, about 4 wt. % or less, about 3 wt. % or less, about 2 wt. % or less, about 1 wt. % or less, about 0.5 wt. % or less, or about 0.1 wt. % or less of a cationic surfactant, based on the total weight of the personal care composition. In some embodiments, the personal care compositions is substantially free or free of cationic surfactants.

The one or more cationic surfactants, if present, may include or be chosen from quaternary ammonium compounds, amidoamines, or a combination of two or more thereof. Examples of cationic surfactants include cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, olcamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, or a combination of two or more thereof.

Additional, non-limiting examples of cationic surfactants include: cetyl trimethyl ammonium chloride available, for example, with trade name CA-2350 from Nikko Chemicals and CTAC 30KC available from KCI, stearyl trimethyl ammonium chloride with trade name Arquad 18/50 available from Akzo Nobel, hydrogenated tallow alkyl trimethyl ammonium chloride, stearyl dimethyl benzyl ammonium chloride, stearyl propyleneglycol phosphate dimethyl ammonium chloride, stearoyl amidopropyl dimethyl benzyl ammonium chloride, stearoyl amidopropyl dimethyl (myristylacetate) ammonium chloride, and N-(stearoyl colamino formyl methyl)pyridinium chloride. Further cationic surfactants that may be included or excluded in certain embodiments include quaterniums, such as quaternium-16, quaternium-26, quaternium-27, quaternium-30, quaternium-33, quaternium-43, quaternium-52, quaternium-53, quaternium-56, quaternium-60, quaternium-61, quaternium-62, quaternium-70, quaternium-71, quaternium-72, quaternium-75, quaternium-76 hydrolyzed collagen, quaternium-77, quaternium-78, quaternium-79 hydrolyzed collagen, quaternium-79 hydrolyzed keratin, quaternium-79 hydrolyzed milk protein, quaternium-79 hydrolyzed silk, quaternium-79 hydrolyzed soy protein, and quaternium-79 hydrolyzed wheat protein, quaternium-80, quaternium-81, quaternium-82, quaternium-83, quaternium-84, or a combination of two or more thereof. Other cationic surfactants worth mentioning include cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, olcamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidocthyldimethylamine, arachidamidopropyldimethylamine, arachidamido-propyidiethylamine, arachidamidoethyidiethylamine, arachidamidoethyidimethylamine, or a combination of two or more thereof.

The personal care compositions may be formulated to have a surfactant system having certain weight ratios of surfactants. Preferably, the personal care composition has a weight ratio of the sulfate based anionic surfactant(s) to the non-sulfate based anionic surfactant(s) ranging from about 6:1 to about 3:2. For example, the personal care compositions may have a weight ratio of the total amount of sulfate based anionic surfactant(s) to the total amount of non-sulfate based anionic surfactant(s) from about 6:1 to about 3:2, about 5:1 to about 3:2, about 4:1 to about 3:2, about 3:1 to about 3:2, about 2:1 to about 3:2, about 1:1 to about 3:2; from about 6:1 to about 1:1, about 5:1 to about 1:1, about 4:1 to about 1:1, about 3:1 to about 1:1, about 2:1 to about 1:1; from about 6:1 to about 2:1, about 5:1 to about 2:1, about 4:1 to about 2:1; from about 6:1 to about 3:1, about 5:1 to about 3:1, or any range or subrange thereof. In some embodiments, the surfactant system comprises alpha olefin sulfonate and the personal care composition is formulated to have a weight ratio of sulfate based anionic surfactants to alpha olefin sulfonate according to any of the weight ratios listed above for the weight ratio of sulfate based anionic surfactant(s) to the non-sulfate based anionic surfactant(s). In further embodiments, the surfactant system comprises an alpha olefin sulfonate and at least one of sodium lauryl sulfate or sodium laureth sulfate, and where the personal care composition is formulated to have a weight ratio of the at least one of sodium lauryl sulfate or sodium laureth sulfate to the alpha olefin sulfonate according to any of the weight ratios listed above for the weight ratio of sulfate based anionic surfactant(s) to the non-sulfate based anionic surfactant(s).

In addition to the surfactant system, the personal care composition typically comprises one or more thickening agent(s). Thickening agents may be referred to as “thickeners” or “viscosity modifying agents.” Thickening agents are typically included to increase the viscosity of the personal care compositions. Nonetheless, in some instances, certain thickening agents provide additional, surprising benefits to the personal care compositions. For instance, the inventors discovered that certain personal care compositions comprising certain surfactant systems described herein and thickening agents (e.g., xanthan gum) in particular amounts unexpectedly achieves superior foaming characteristics and certain rheological properties. Without being limited to any specific theory, it was discovered that particular embodiments of personal care compositions having surfactant systems disclosed herein and certain thickening agents in an amount, such that upon dilution, the thickening was present in an amount from about 0.005 to about 0.05 wt. % yield surprisingly beneficial foaming characteristics and rheological properties.

The amount of thickening agent in the personal care composition may vary depending, e.g., on the form of the personal care composition (diluted or non-diluted personal care composition), the desired foaming properties, and/or desired rheological properties. The personal care composition prior to dilution (e.g. in a non-diluted and/or concentrated form) may be formulated to have one or more thickening agent(s) in an amount from about 0.05 to about 5 wt. %, based on the total weight of the personal care composition. For instance, the total amount of thickening agent(s) in the non-diluted personal care composition may be from about 0.05 to about 4 wt. %, about 0.05 to about 3 wt. %, about 0.05 to about 2 wt. %, about 0.05 to about 1 wt. %, about 0.05 to about 0.5 wt. %, about 0.05 to about 0.3 wt. %; from about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.5 wt. %, about 0.1 to about 0.3 wt. %; from about 0.2 to about 5 wt. %, about 0.2 to about 4 wt. %, about 0.2 to about 3 wt. %, about 0.2 to about 2 wt. %, about 0.2 to about 1 wt. %, about 0.2 to about 0.5 wt. %; from about 0.5 to about 5 wt. %, about 0.5 to about 4 wt. %, about 0.5 to about 3 wt. %, about 0.5 to about 2 wt. %, about 0.5 to about 1 wt. %; from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %, or any range or subrange thereof, based on the total weight of the personal care composition.

The personal care composition when in a diluted form (also referred herein as diluted personal care composition) and/or ready-to-use form may have the one or more thickening agents present in an amount from about 0.005 to about 0.05 wt. %, based on the total weight of the diluted personal care composition. In some embodiments, the total amount of thickening agent(s) present in the diluted personal care composition is from about 0.005 to about 0.04 wt. %, about 0.005 to about 0.03 wt. %, about 0.005 to about 0.02 wt. %, about 0.005 to about 0.01 wt. %; from about 0.007 to about 0.05 wt. %, about 0.007 to about 0.04 wt. %, about 0.007 to about 0.03 wt. %, about 0.007 to about 0.02 wt. %, about 0.007 to about 0.01 wt. %; from about 0.009 to about 0.05 wt. %, about 0.009 to about 0.04 wt. %, about 0.009 to about 0.03 wt. %, about 0.009 to about 0.02 wt. %; from about 0.01 to about 0.05 wt. %, about 0.01 to about 0.04 wt. %, about 0.01 to about 0.03 wt. %, about 0.01 to about 0.02 wt. %; from about 0.02 to about 0.05 wt. %, about 0.02 to about 0.04 wt. %, about 0.02 to about 0.03 wt. %; from about 0.03 to about 0.05 wt. %, about 0.03 to about 0.04 wt. %, about 0.04 to about 0.05 wt. %, or any range or subrange thereof, based on the total weight of the diluted personal care composition.

The one or more thickening agent(s) may be selected from natural gums, polyacrylate crosspolymers or crosslinked polyacrylate polymers, cationic acrylate copolymers, anionic acrylic or carboxylic acid polymers, polyacrylamide polymers, polysaccharides such as cellulose derivatives, cationic gums, polyquaterniums, vinylpyrrolidone homopolymers/copolymers, C₈₋₂₄ hydroxyl substituted aliphatic acid, C₈₋₂₄ conjugated aliphatic acid, sugar fatty esters, polyglyceryl esters, and a mixture thereof. Particular types of thickening agents that may be mentioned include the following: gums (e.g., natural gums), sucrose esters, polyvinylpyrrolidone (PVP) and co-polymers, celluloses, polyquaternium compounds, and carboxylic acid or carboxylate based homopolymer or co-polymer, which can be linear or crosslinked. In at least one preferred embodiment, the thickening agent comprises xanthan gum.

The personal care compositions may include one or more fatty ester(s). The amount of fatty ester in the personal care composition may be from about 0.5 to about 25 wt. %, based on the total weight of the personal care composition. For example, the total amount of fatty ester(s) in the personal care composition may be from about 0.5 to about 20 wt. %, about 0.5 to about 16 wt. %, about 0.5 to about 12 wt. %, about 0.5 to about 10 wt. %, about 0.5 to about 8 wt. %, about 0.5 to about 7 wt. %, about 0.5 to about 6 wt. %, about 0.5 to about 5 wt. %, about 0.5 to about 4 wt. %, about 0.5 to about 3 wt. %; from about 1 to about 25 wt. %, about 1 to about 20 wt. %, about 1 to about 16 wt. %, about 1 to about 12 wt. %, about 1 to about 10 wt. %, about 1 to about 8 wt. %, about 1 to about 7 wt. %, about 1 to about 6 wt. %, about 1 to about 5 wt. %, about 1 to about 4 wt. %, about 1 to about 3 wt. %; from about 2 to about 25 wt. %, about 2 to about 20 wt. %, about 2 to about 16 wt. %, about 2 to about 12 wt. %, about 2 to about 10 wt. %, about 2 to about 8 wt. %, about 2 to about 7 wt. %, about 2 to about 6 wt. %, about 2 to about 5 wt. %, about 2 to about 4 wt. %, about 2 to about 3 wt. %; from about 3 to about 25 wt. %, about 3 to about 20 wt. %, about 3 to about 16 wt. %, about 3 to about 12 wt. %, about 3 to about 10 wt. %, about 3 to about 8 wt. %, about 3 to about 7 wt. %, about 3 to about 6 wt. %, about 3 to about 5 wt. %, about 3 to about 4 wt. %; from about 4 to about 25 wt. %, about 4 to about 20 wt. %, about 4 to about 16 wt. %, about 4 to about 12 wt. %, about 4 to about 10 wt. %, about 4 to about 8 wt. %, about 4 to about 7 wt. %, about 4 to about 6 wt. %; from about 6 to about 25 wt. %, about 6 to about 20 wt. %, about 6 to about 16 wt. %, about 6 to about 12 wt. %, about 6 to about 10 wt. %; from about 8 to about 25 wt. %, about 8 to about 20 wt. %, about 8 to about 16 wt. %, about 8 to about 12 wt. %, or any range or subrange thereof, based on the total weight of the personal care composition.

The personal care compositions may include one or more fatty ester(s) that are chosen from polyglycerol esters of fatty acids, glycerol fatty esters, fatty carbonate esters, esters of fatty acids and glucose or alkylglucose, sucrose fatty esters, fatty carbonate ester, sorbitan fatty ester, or mixtures of two or more thereof. In some cases, the fatty ester may be chosen from glycerol fatty esters, such as those selected from glyceryl monomyristate, glyceryl monopalmitate, glyceryl monostearate, glyceryl isostearate, glyceryl monooleate, glyceryl dioleate, glyceryl distearate, glyceryl laurate, trilaurin, triarachidin, tribehenin, tricaprin, tricaprylin, caprylic/capric triglyceride, tricrucin, triheptanoin, triheptylundccanoin, triisononanoin, triisopalmitin, triisostearin, trilinolein, trimyristin, trioctanoin, triolein, tripalmitin, tripalmitolein, triricinolein, tristearin, triundecanoin, and mixtures of two or more thereof.

Preferably, the one or more fatty ester(s) is a glycerol fatty ester and/or polyglycerol ester of fatty acids. Examples of polyglycerol esters of fatty acids include polyglyceryl-4 caprylate/caprate, polyglyceryl-10 caprylate/caprate, polyglyceryl-4 caprate, polyglyceryl-10 caprate, polyglyceryl-4 laurate, polyglyceryl-5 laurate, polyglyceryl-6 laurate, polyglyceryl-10 laurate, polyglyceryl-10 cocoate, polyglyceryl-10 myristate, polyglyceryl-10 oleate, polyglyceryl-10 stearate, and mixtures thereof.

Without being limited to any specific theory, the inventors discovered that utilizing particular surfactant systems and thickener(s) described herein with certain fatty esters surprisingly achieved a personal care composition having improved transparency and increased stability. The one or more fatty ester(s) may in some embodiments comprise polyglyceryl caprates. In at least one embodiment, the personal care composition includes polyglyceryl caprate(s), such as those selected from polyglyceryl-2-caprate, polyglyceryl-4-caprate, polyglyceryl-6-caprate, and a combination of two or more thereof.

Additional non-limiting examples of fatty esters include fatty esters, such as esters of C₆₋₂₂ fatty acids with a monohydric alcohol and/or esters of C₆₋₂₂ fatty alcohols with a monocarboxylic acid. More non-limiting examples include isopropyl isostearate, n-propyl myristate, isopropyl myristate, hexyl laurate, hexadecyl isostearate, hexydecyl laurate, hexyldecyl octanoate, n-propyl palmitate, isopropyl palmitate, and mixtures thereof.

The personal care composition may include one or more polyol(s). Typically, the personal care compositions include the one or more polyol(s) in an amount from about 3 to about 25 wt. %, based on the total weight of the personal care composition. For example, the total amount of polyol(s) in the personal care composition may be from about 3 to about 22 wt. %, about 3 to about 20 wt. %, about 3 to about 18 wt. %, about 3 to about 16 wt. %; from about 5 to about 25 wt. %, about 5 to about 22 wt. %, about 5 to about 20 wt. %, about 5 to about 18 wt. %, about 5 to about 16 wt. %; from about 7 to about 25 wt. %, about 7 to about 22 wt. %, about 7 to about 20 wt. %, about 7 to about 18 wt. %, about 7 to about 16 wt. %; from about 9 to about 25 wt. %, about 9 to about 22 wt. %, about 9 to about 20 wt. %, about 9 to about 18 wt. %, about 9 to about 16 wt. %; from about 12 to about 25 wt. %, about 12 to about 22 wt. %, about 12 to about 20 wt. %, about 12 to about 18 wt. %, about 12 to about 16 wt. %; from about 14 to about 25 wt. %, about 14 to about 22 wt. %, about 14 to about 20 wt. %, about 14 to about 18 wt. %, from about 16 to about 25 wt. %, about 16 to about 22 wt. %, about 16 to about 20 wt. %; from about 18 to about 25 wt. %, about 21 to about 25 wt. %, or any range or subrange thereof, based on the total weight of the personal care composition.

The polyol(s) may be chosen from glycols or compounds with numerous hydroxyl groups. In some cases, the one or more polyols is/are selected from the group consisting of C₂-C₃₂ polyols. The one or more polyols may be liquid at ambient temperature (25° C.). The one or more polyols may have from 2 to 32 carbon atoms, from 3 to 16 carbon atoms, or from 3 to 12 carbon atoms.

Polyols that may be included in the personal care composition, in certain instances, include ethylene glycol, propylene glycol, butylene glycol, hexylene glycol, glycerin, diglycerin, diethylene glycol, dipropylene glycol, and mixtures thereof. In some cases, the personal care composition comprises glycerin, and optionally one or more polyols other than glycerin. Non-limiting examples of polyols that may, optionally, be included in the personal care include alkanediols such as glycerin, 1,2,6-hexanetriol, trimethylolpropane, ethylene glycol, propylene glycol, butylene glycol, pentylene glycol, hexylene glycol, diethylene glycol, tricthylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, or a combination of two or more thereof. In at least one embodiment, the personal care composition includes one or more polyol(s) comprising panthenol, pantothenic acid, or a combination thereof.

The one or more polyols may, optionally, be glycols or glycol ethers such as, e.g., monomethyl, monoethyl and monobutyl ethers of ethylene glycol, propylene glycol or ethers thereof such as, e.g., monomethyl ether of propylene glycol, butylene glycol, hexylene glycol, dipropylene glycol as well as alkyl ethers of diethylene glycol, e.g., monoethyl ether or monobutyl ether of diethylene glycol. In some cases, the one or more polyols may include ethylene glycol, propylene glycol, butylene glycol, hexylene glycol, pentylene glycol, 1,3-propanediol, diethylene glycol, dipropylene glycol, 1,4-butanediol, 1,5-pentanediol, hexane-1,6-diol, glycerin, diglycerin, caprylyl glycol, or a combination of two or more thereof. In certain embodiments, the personal care composition may include polyol(s) selected from glycerin (preferably, vegetable refined glycerin), butylene glycol, and 1,3-propanediol, polyglutamic acid, saccharide isomerate, and combinations thereof. In some embodiments, the polyol is a polyethylene glycol, such as polyethylene glycol 600.

Additionally or alternatively, the personal care composition may include polyol(s) having a molecular weight of from about 100 to 5000 g/mol. For instance, the polyol may comprise a polyethylene glycol, a polypropylene glycol, a block polymer of polyethylene glycol and polypropylene glycol, or a combination of two or more thereof. In some embodiments, the polyol comprises a polypropylene glycol, a polypropylene glycol, and/or a block polymer of polyethylene glycol and polypropylene glycol having a molecular weight of about 100 to about 900, about 200 to about 800, about 400, about 1500 to about 2500, about 2000 to about 4500 or any range or subrange thereof.

The personal care composition may, in some instances, include water, e.g., in an amount from about 0.5 to about 40 wt. %, based on the total weight of the personal care composition. For example, the amount of water present in the personal care composition may be from about 0.5 to about 36 wt. %, about 0.5 to about 32 wt. %, about 0.5 to about 28 wt. %, about 0.5 to about 23 wt. %, about 0.5 to about 17 wt. %, about 0.5 to about 14 wt. %, about 0.5 to about 11 wt. %, about 0.5 to about 8 wt. %, about 0.5 to about 6 wt. %, about 0.5 to about 4 wt. %, about 0.5 to about 2 wt. %; from about 2 to about 40 wt. %, about 2 to about 36 wt. %, about 2 to about 32 wt. %, about 2 to about 28 wt. %, about 2 to about 23 wt. %, about 2 to about 17 wt. %, about 2 to about 14 wt. %, about 2 to about 11 wt. %, about 2 to about 8 wt. %, about 2 to about 6 wt. %; from about 5 to about 40 wt. %, about 5 to about 36 wt. %, about 5 to about 32 wt. %, about 5 to about 28 wt. %, about 5 to about 23 wt. %, about 5 to about 17 wt. %, about 5 to about 14 wt. %, about 5 to about 11 wt. %; from about 10 to about 40 wt. %, about 10 to about 36 wt. %, about 10 to about 32 wt. %, about 10 to about 28 wt. %, about 10 to about 23 wt. %, about 10 to about 17 wt. %; from about 15 to about 40 wt. %, about 15 to about 36 wt. %, about 15 to about 32 wt. %, about 15 to about 28 wt. %, about 15 to about 23 wt. %, from about 20 to about 40 wt. %, about 20 to about 36 wt. %, about 20 to about 32 wt. %, about 20 to about 28 wt. %; from about 25 to about 40 wt. %, about 25 to about 36 wt. %, about 25 to about 32 wt. %, or any range or subrange thereof, based on the total weight of the personal care composition.

The personal care composition may further comprise one or more colorants. The colorants may be a pigment, a dye, or mixtures thereof. Non-limiting examples of pigments include titanium dioxide, zinc oxide, kaolin, mica etc. Non-limiting examples of dyes include food dyes suitable for food, drug and cosmetic applications, and mixtures thereof. Some color agents (colorants) are known as FD&C dyes. In some embodiments, the colorants may be present in an amount ranging from about 0.0001 to about 0.4 wt. %, including all percentages and subranges thereof, based on the total weight of the personal care composition. In further embodiments, the colorants may be present in an amount ranging from about 0.0001 to about 4 wt. %, including all percentages and subranges thereof, based on the total weight of the personal care composition.

The personal care composition may include one or more pH adjusters to increase or decrease the overall pH of the personal care composition. For example, one or more acids may be included to decrease the pH of the personal care composition. Examples of suitable acids for decreasing the pH of the personal care composition include, but are not limited to, citric acid, acetic acid, and the like. The personal care composition may include one or more bases, such as sodium hydroxide, potassium hydroxide and the like, to increase the pH of the personal care composition. Additional or alternative acids and bases that are suitable for adjusting the pH of the personal care composition are readily known to one of ordinary skill in the art.

The amount of the pH adjuster in the personal care composition may be based on the desired pH of the final form of personal care composition and/or product (e.g., in a non-diluted/concentrated form and/or in a diluted/ready-to-use form). For example, the total amount of the pH adjuster may range from about 0.05 to about 20 wt. %, based on the total weight of the personal care composition. In some instances, the total amount of pH adjuster is from about 0.05 to about 15 wt. %, about 0.1 to about 10 wt. %, or about 0.12 to about 5 wt. %, including ranges and sub-ranges therebetween, based on the total weight of the personal care composition.

The personal care compositions may have a pH from 4.5 to about 10, 4.5 to about 9, 4.5 to about 8, 4.5 to about 7, 4.5 to about 6; from about 5 to about 10, about 5 to about 9, about 5 to about 8, about 5 to about 7, about 5 to about 6; from about 6 to about 10, about 6 to about 9, about 6 to about 8, or about 6 to about 7; from about 7 to about 10, about 7 to about 9, or about 7 to about 8, including any ranges and subranges therebetween.

The personal care compositions may, in some cases, comprise one or more preservatives. The personal care composition may be formulated to have preservative(s) in an amount from about 0.1 to about 25 wt. %, based on the total weight of the personal care composition. For example, the preservative(s) may be present in the personal care composition in an amount from about 0.1 to about 22 wt. %, about 0.1 to about 19 wt. %, about 0.1 to about 16 wt. %, about 0.1 to about 14 wt. %, about 0.1 to about 12 wt. %, about 0.1 to about 10 wt. %, about 0.1 to about 8 wt. %, about 0.1 to about 6 wt. %, about 0.1 to about 4 wt. %, about 0.1 to about 3 wt. %, about 0.1 to about 2 wt. %, about 0.1 to about 1 wt. %; from about 1 to about 25 wt. %, about 1 to about 22 wt. %, about 1 to about 19 wt. %, about 1 to about 16 wt. %, about 1 to about 14 wt. %, about 1 to about 12 wt. %, about 1 to about 10 wt. %, about 1 to about 8 wt. %, about 1 to about 6 wt. %, about 1 to about 4 wt. %, about 1 to about 3 wt. %; from about 2 to about 25 wt. %, about 2 to about 22 wt. %, about 2 to about 19 wt. %, about 2 to about 16 wt. %, about 2 to about 14 wt. %, about 2 to about 12 wt. %, about 2 to about 10 wt. %, about 2 to about 8 wt. %, about 2 to about 6 wt. %, about 2 to about 4 wt. %, about 2 to about 3 wt. %; from about 3 to about 25 wt. %, about 3 to about 22 wt. %, about 3 to about 19 wt. %, about 3 to about 16 wt. %, about 3 to about 14 wt. %, about 3 to about 12 wt. %, about 3 to about 10 wt. %, about 3 to about 8 wt. %, about 3 to about 6 wt. %, about 3 to about 4 wt. %; from about 5 to about 25 wt. %, about 5 to about 22 wt. %, about 5 to about 19 wt. %, about 5 to about 16 wt. %, about 5 to about 14 wt. %, about 5 to about 12 wt. %, about 5 to about 10 wt. %, about 5 to about 8 wt. %; from about 7 to about 25 wt. %, about 7 to about 22 wt. %, about 7 to about 19 wt. %, about 7 to about 16 wt. %, about 7 to about 14 wt. %, about 7 to about 12 wt. %; from about 10 to about 25 wt. %, about 10 to about 22 wt. %, about 10 to about 19 wt. %, about 10 to about 16 wt. %, about 10 to about 14 wt. %; from about 13 to about 25 wt. %, about 13 to about 22 wt. %, about 13 to about 19 wt. %; from about 15 to about 25 wt. %, about 15 to about 22 wt. %, about 15 to about 19 wt. %; from about 17 to about 25 wt. %, about 17 to about 22 wt. %, or any range or subrange thereof, based on the total weight of the personal care composition.

The one or more preservatives may include at least one of lactic acid, benzalkonium chloride, sodium benzoate, potassium sorbate, hydroxyacetophenone, ethylhexylglycerin, or a combination of thereof. Additional examples of preservatives that may incorporated in the preservative system include, but are not limited to, 4-hydroxy benzoic acid, benzoic acid, sorbic acid, dehydroacetic acid, triclosan, benzyl alcohol, chlorophenesin, salicylic acid, hydroxy acetophenone, caprylhydroxamic acid, phenethyl alcohol, p-anisic acid, glyceryl caprylate, levulinic acid, sodium levulinate, propanediol, hexanediol, pentylene glycol, capryloyl glycine, methylpropanediol, phenylpropanol, sodium anisate, or a combination of two or more thereof.

The preservative system may, in some cases, include or exclude one or more of the following preservatives: chlorhexidine, chlorhexidine digluconate, chlorhexidine dihydrochloride, chlorhexidine diacetate, chlorhexidine gluconate, chlorhexidine hydrochloride, chlorhexidine phosphanilate, chlorphensin, benzoic acid or a salt or ester thereof (e.g. sodium benzoate), propionic acid or a salt thereof, salicylic acid or a salt thereof, sorbic acid or a salt thereof (e.g. potassium sorbate), formaldehyde, paraformaldehyde, zinc pyrithione, inorganic sulphites, hydrogen sulphites, chlorobutanol, 4-hydroxybenzoic acid or a salt or ester thereof (e.g. methylparaben, ethylparaben, propylparaben), dehydroacetic acid and/or a salt thereof (e.g. sodium dehydroacetate), formic acid or a salt thereof, dibromohexamidine isethionate; thimerosal, phenylmercuric salts, undecylenic acid or a salt thereof, hexetidine, bronopol, 5-bromo-5-nitro-1,3-dioxane, dichlorobenzyl alcohol, benzyl alcohol, triclocarban, chlororesol, triclosan, chloroxylenol, imidazolidinyl urea, polyaminopropyl biguanide, phenoxyethanol, methenamine, quaternium-15, climbazole, DMDM hydantoin, 1-hydroxy-4-methyl-6-(2,4,4-trimethylenepentyl)-2 pyridon, piroctone olamine, bromochlorophene, 0-cymen-5-ol, methylchloroisothiazolinone, methylisothiazololinone, mixtures of methylchloroisothiazolinone and methylisothiazololinone, chlorophene, chloroacetamide, phenoxyisoproponol, alkyl (C12-C22) trimethyl ammonium bromide, alkyl (C12-C22) trimethyl ammonium chloride, dimethyl oxazolidine, diazolidinyl urea, hexamidine, hexamidine diisethionate, hexamidine diparaben, hexamidine paraben, glutaral, 7-ethylbicyclooxazolidine, sodium hydroxymethylaminoacetate, silver chloride, benzethonium chloride, benzalkonium chloride, benzalkonium bromide, benzalkonium saccharinate, benzylhemiformal, iodopropynyl butylcarbamate, silver citrate, or a combination of two or more thereof.

As discussed above, a personal care composition when in a diluted form (also referred herein as diluted personal care composition) and/or ready-to-use form may be produced by diluting the personal care composition with an amount of water. Although the personal care compositions may be diluted to produce a diluted personal care composition having about 0.005 to about 0.05 wt. % of thickening agent(s), based on the total weight of the diluted personal care composition, the personal care composition may be diluted with water at a weight ratio of about 1:0.5 to about 1:10. For instance, the personal care composition may be diluted with water at a weight ratio (personal care composition: water) of about 1:0.5 to about 1:10, about 1:0.5 to about 1:8, about 1:0.5 to about 1:6, about 1:0.5 to about 1:4, about 1:0.5 to about 1:2, about 1:0.5 to about 1:1; from about 1:1 to about 1:10, about 1:1 to about 1:8, about 1:1 to about 1:6, about 1:1 to about 1:4, about 1:1 to about 1:2; from about 1:2 to about 1:10, about 1:2 to about 1:8, about 1:2 to about 1:6, about 1:2 to about 1:4; from about 1:3 to about 1:10, about 1:3 to about 1:8, about 1:3 to about 1:6, about 1:3 to about 1:4; from about 1:4 to about 1:10, about 1:4 to about 1:8, about 1:4 to about 1:6, or any range or subrange thereof.

According to another aspect of the invention, the personal care composition may be encapsulated in a pod. The encapsulated personal care composition may include any of the components and/or ingredients described herein. The personal care composition encapsulated in the pod is typically not in a diluted form and/or is in a concentrated form.

The pod is generally formed from a film that may encapsulate and/or surround the personal care composition. In some instance, the pod is formed from two films that are coupled together, e.g., along one or more edges. When the pod is formed of two, three, or fourth films, each of the films may be the same or different.

The film is generally formed from a monomer or polymer that dissolves in water. Preferably, the film of the pod is formed of hydroxypropyl methylcellulose (HPMC), polyvinyl alcohol (PVA), or a combination of two or more thereof. In at least one embodiment, the film of the pod comprises a polymer of HPMC and PVA.

A diluted and/or ready-to-use personal care composition may be formed by combining one or more pod(s) containing the personal care composition with water to dissolve the film of the pod, thereby releasing the personal care composition. For example, the pod containing the personal care composition may be placed in a soap dispenser with water to dissolve the film of the pod and produce a diluted and/or ready-to-use form of the personal care composition.

Preferably, the film of the pod dissolves in an amount of time from about 1 second to about 60 seconds. In some embodiments, the film of the pod dissolves in an amount of time from about 1 to about 50 seconds, about 1 to about 40 seconds, about 1 to about 30 seconds, about 1 to about 25 seconds, about 1 to about 20 seconds, about 1 to about 15 seconds, about 1 to about 10 seconds; from about 3 to about 60 seconds, about 3 to about 50 seconds, about 3 to about 40 seconds, about 3 to about 30 seconds, about 3 to about 25 seconds, about 3 to about 20 seconds, about 3 to about 15 seconds, about 3 to about 10 seconds; from about 5 to about 60 seconds, about 5 to about 50 seconds, about 5 to about 40 seconds, about 5 to about 30 seconds, about 5 to about 25 seconds, about 5 to about 20 seconds, about 5 to about 15 seconds; from about 10 to about 60 seconds, about 10 to about 50 seconds, about 10 to about 40 seconds, about 10 to about 30 seconds, about 10 to about 25 seconds, about 10 to about 20 seconds; from about 15 to about 60 seconds, about 15 to about 50 seconds, about 15 to about 40 seconds, about 15 to about 30 seconds, about 15 to about 25 seconds, or any range or subrange thereof. The amount of time for dissolving the pod may be based on agitating the water (e.g., stirring, shaking, swirling, or the like) the water and pod. In some embodiments, however, the amount of time for dissolving the pod is based on the water and pod not being agitated.

The personal care composition may be formulated such that the viscosity increases when the personal care composition is diluted with water at a weight ratio of about 1:0.5 to about 1:10. For instance, the viscosity of the personal care composition may increase when diluted with water at a weight ratio of about 1:0.5 to about 1:10, about 1:0.5 to about 1:8, about 1:0.5 to about 1:6, about 1:0.5 to about 1:4, about 1:0.5 to about 1:2, about 1:0.5 to about 1:1; from about 1:1 to about 1:10, about 1:1 to about 1:8, about 1:1 to about 1:6, about 1:1 to about 1:4, about 1:1 to about 1:2; from about 1:2 to about 1:10, about 1:2 to about 1:8, about 1:2 to about 1:6, about 1:2 to about 1:4; from about 1:3 to about 1:10, about 1:3 to about 1:8, about 1:3 to about 1:6, about 1:3 to about 1:4; from about 1:4 to about 1:10, about 1:4 to about 1:8, about 1:4 to about 1:6, or any range or subrange thereof.

In some embodiments, the weight ratio of the sulfate based anionic surfactant to the non-sulfate based anionic surfactant is from about 6:1 to about 2:1 or, optionally, from about 5:1 to about 3:1. In other embodiments, the surfactant system is present in an amount from about 30 to about 85 wt. %; optionally, from about 35 to about 85 wt. %, from about 40 to about 85 wt. %, from about 45 to about 85 wt. %, from about 50 to about 80 wt. %, from about 50 to about 75 wt. %, or from about 50 to about 70 wt. %. In certain embodiments, the sulfate based anionic sulfate comprises sodium lauryl sulfate, sodium laureth sulfate, or a combination of two or more thereof. In further embodiments, the non-sulfate based anionic surfactant is selected from acyl sulfates, acyl isethionates, acyl amino acids, acyl sarcosinates, acyl taurates, acyl glycinates, acyl glutamates, alkyl sulfonates, alkyl sulfosuccinates, alkyl sulfoacetates, alkoxylated monoacids, and a combination of two or more thereof.

In some embodiments, the non-sulfate based anionic surfactant comprises sodium olefin sulfonate. In certain embodiments, the sulfate based anionic surfactant is present in an amount from about 20 to about 70 wt. %; optionally, from about 30 to about 70 wt. %, from about 40 to about 70 wt. %, from about 45 to about 65 wt. %, or from about 45 to about 60 wt. %. In other embodiments, the non-sulfate based anionic surfactant is present in an amount from about 5 to about 20 wt. %; optionally, from about 7 to about 20 wt. %, from about 10 to about 20 wt. %, from about 10 to about 17, or from about 10 to about 15 wt. %.

In some embodiments, the personal care composition is substantially free, or free, of water. In some embodiments, the personal care composition is in the form of a tablet, optionally a powder tablet.

Some embodiments of the present invention provide personal care compositions comprising a fatty ester comprising a polyglyceryl caprate. In some embodiments, the polyglyceryl caprate is selected from polyglyceryl-2-caprate, polyglyceryl-4-caprate, polyglyceryl-6-caprate, and a combination of two or more thereof. In certain embodiments, the personal care composition has less than about 1 ppm of 1,4 dioxane.

Other embodiments provide personal care compositions comprising a polyol selected from panthenol, pantothenic acid, and a combination thereof.

### EXAMPLES

### Example 1

An exemplary personal care composition (Ex. A) was prepared in accordance with aspects of the invention. Eight comparative compositions (Comps. 1-8) were prepared having formulations similar to Ex. A. Ex. A and Comps. 1-8 were in the form of powder compositions, which were encapsulated in a pod. The formulations of Ex. A and Comps. 1-8 are shown in Table 1.

TABLE 1

| | Ex. A | Comp. 1 | Comp. 2 | Comp. 3 | Comp. 4 | Comp. 5 | Comp. 6 | Comp. 7 | Comp. 8 |
|---|---|---|---|---|---|---|---|---|---|
| INCI Compound Name | (g) | (g) | (g) | (g) | (g) | (g) | (g) | (g) | (g) |
| Sodium Lauryl Sulfate | 6.8 | 6.8 | 8.8 | 6.8 | 6.8 | 6.8 | 6.8 | 6.8 | 6.8 |
| Panthenol | 0.5 | 0.5 | 0.5 | 0.5 | 0.5 | 0.5 | 0.5 | 0.5 | 0.5 |
| Sorbitol | 1.5 | 1.5 | 1.5 | 1.5 | 1.5 | 1.5 | 1.5 | 1.5 | 1.5 |
| Phenoxyethanol, Caprylyl | 2.5 | 2.5 | 2.5 | 2.5 | 2.5 | 2.5 | 2.5 | 2.5 | 2.5 |
| Glycol, Decylene Glycol | | | | | | | | | |
| Outdoor Fizz Compact | 0.23 | 0.23 | 0.23 | 0.23 | 0.23 | 0.23 | 0.23 | 0.23 | 0.23 |
| Sodium Lauroyl Sarcosinate | | | | 2 | | | | | |
| Sodium C14-16 Olefin | | | | | 2 | | | | |
| Sulfonate | | | | | | | | | |
| PVP | | | | | | 2 | | | |
| Cocamidopropy 1 Betaine | | | | | | | 2 | | |
| Xanthan Gum | 0.03 | | | | | | | | |
| Poloxamer 188 | | | | | | | | 2 | |
| Poloxomer 407 | | | | | | | | | 2 |
| Water | 2 | 2 | | | | | | | |
| Total (g) | 13.5 | 13.5 | 13.5 | 13.5 | 13.5 | 13.5 | 13.5 | 13.5 | 13.5 |

### Example 2

Ex. A and Comps. 1-8 were evaluated to assess the rate of dilution and the foam characteristics of such personal care compositions. Specifically, Ex. A and Comps. 1-8 were diluted with 240 grams of water to form ready to use, diluted compositions. Each of the respective diluted compositions was monitored to determine the amount of time required to completely dissolve each of Ex. A and Comps. 1-8 in the respective diluted compositions. Specifically, the dilution times were determined as the time between when the mixture was added to when a homogenous solution was achieved.

The amount of time to completely dissolve each of Ex. A and Comps. 1-8 to form a homogenous diluted composition is shown in Table 2, below.

TABLE 2

| Personal Care | |
|---|---|
| Composition | Dissolution Time |
| Ex. A | about 5 seconds |
| Comp. 2 | about 3 minutes |
| Comp. 3 | about 1 minute |
| Comp. 4 | about 2 minutes, |
| Comp. 5 | about 1.5 minutes |
| Comp. 6 | about 10 minutes |
| Comp. 7 | about 9 minutes |
| Comp. 8 | about 30 minutes |

To assess the foam characteristics, 7.5 grams of each diluted composition was further diluted with 50 grams of tap water to mimic a soap to water ratio similar to a typical hand washing experience. A dynamic foam analyzer (DFA) instrument was subsequently used to measure the foam characteristics produced from the diluted compositions of Ex. A and Comps. 1-8, including the maximum foam volume, foam half-life, bubble count, and bubble area to primarily gauge foaming performance. The DFA instrument generated the foam by stirring the mixture using a metal stir bar.

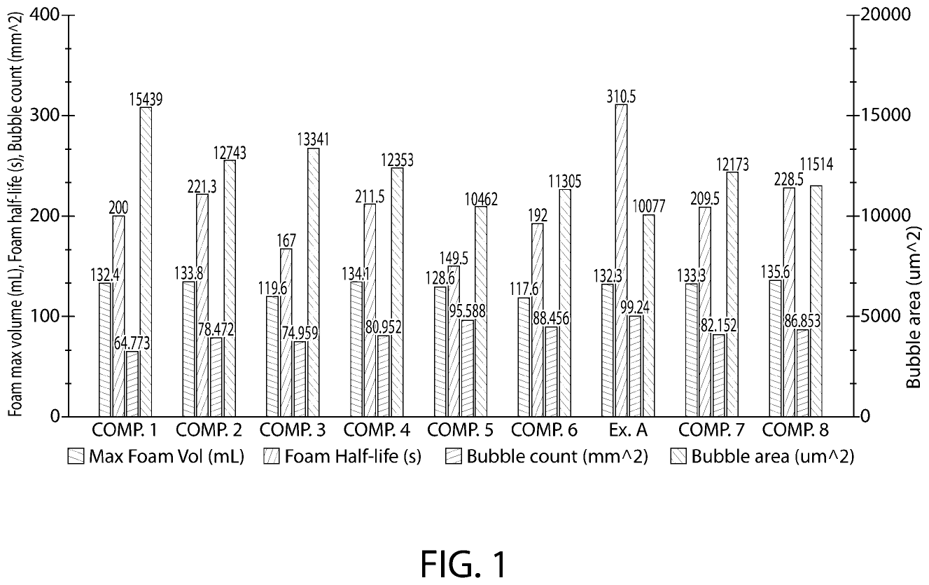

Based on the DFA data, it was determined that maximum foam volume was increased with the use of sodium C₁₄₋₁₆ olefin sulfonate, sodium lauryl sulfate, and one of poloxomer 407 and poloxamer 188. However, the amount of time for Comps. 7 and 8 to dissolve in warm water to form a ready to use diluted composition was about 9 minutes or about 30 minutes, respectively, which is highly undesirable. Ex. A surprisingly exhibited a high maximum foam and significantly superior foam half-life and bubble count compared to Comps. 1-8, while having a dilution time of about 5 seconds. FIG. 1 provides the results for the maximum foam volume, foam half-life, bubble count, and bubble area of Ex. A and Comps. 1-8.

### Example 3

Four example compositions (Exs. B-E) containing sodium C₁₄₋₁₆ olefin sulfonate, sodium lauryl sulfate, and xanthan in accordance with aspects of the invention. Eight comparative compositions (Comps. 9-16) were prepared having similar formulations as Exs. B-E. The formulations for Exs. B-E and are shown in Table 3 (below). Comparative Compositions 9-16 are shown in Table 4 (below).

TABLE 3

| INCI Compound Name | Ex. B | Ex. C | Ex. D | Ex. E |
|---|---|---|---|---|
| Sodium Lauryl Sulfate | 9 | 18 | 27 | 36 |
| Sodium C14-16 Olefin Sulfonate | 36 | 27 | 18 | 9 |
| Sodium Bicarbonate | 13 | 13 | 13 | 13 |
| Citric Acid | 32 | 32 | 32 | 32 |
| Sodium Benzoate | 11 | 11 | 11 | 11 |
| Xanthan Gum | 0.27 | 0.27 | 0.27 | 0.27 |

TABLE 4

| | Comp. | Comp. | Comp. | Comp. | Comp. | Comp. | Comp. | Comp. |
|---|---|---|---|---|---|---|---|---|
| INCI Compound Name | 9 | 10 | 11 | 12 | 13 | 14 | 15 | 16 |
| Sodium Lauryl Sulfate | | | 9 | 18 | 27 | 36 | 45 | 45 |
| Sodium C14-16 | 45 | 45 | 36 | 27 | 18 | 9 | | |
| Olefin Sulfonate | | | | | | | | |
| Sodium Bicarbonate | 13 | 13 | 13 | 13 | 13 | 13 | 13 | 13 |
| Citric Acid | 32 | 32 | 32 | 32 | 32 | 32 | 32 | 32 |
| Sodium Benzoate | 11 | 11 | 11 | 11 | 11 | 11 | 11 | 11 |
| Xanthan Gum | | 0.27 | | | | | | 0.27 |

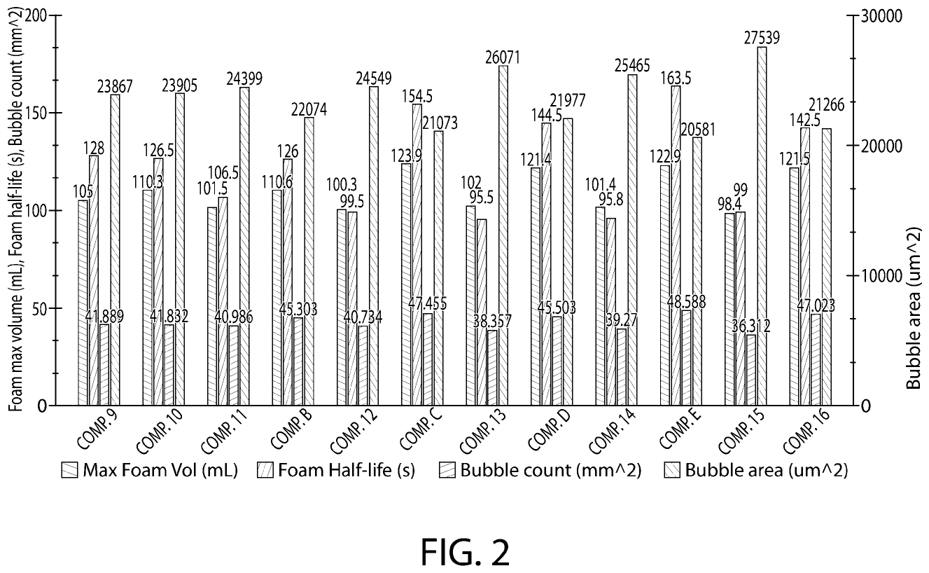

The foam characteristics of Exs. B-E and Comps. 9-16 were evaluating using a DFA instrument using a procedure similar to that described in Example 2. Specifically, the DFA instrument was used to measure the foam characteristics, including the maximum foam volume, foam half-life, bubble count, and bubble area to primarily gauge foaming performance. FIG. 2 provides the results for the maximum foam volume, foam half-life, bubble count, and bubble area of Exs. B-E and Comps. 9-16.

### Example 4

Two exemplary personal care compositions (Ex. F and Ex. G) were prepared in accordance with aspects of the invention. The formulations for Ex. B and Ex. C are shown in Table 5 (below).

TABLE 5

| | Ex. F | Ex. G |
|---|---|---|
| US INCI Name | (wt. %) | (wt. %) |
| Sodium Lauryl Sulfate | 11.2 | 13.4 |
| Sodium Laureth Sulfate | 6.4 | 6.7 |
| Total Anionic Surfactants | 20.3 | 22.9 |
| Cocamidopropyl Betaine | 14.6 | 13.4 |
| Cocamide DIPA | 16.5 | 15.8 |
| Total Surfactants | 67.8 | 67.2 |
| Hydroxyacetophenone | 4.1 | 3.6 |
| Caprylyl Glycol | 2.4 | 2.1 |
| PEG-150 Pentaerythrityl | 2.8 | 6.1 |
| Tetrastearate (and) PPG-2 | | |
| Hydroxyethyl Cocamide | | |
| (and) Aqua | | |
| PEG-40 | 0.9 | 0.4 |
| Propylene Glycol | 10.9 | 9.5 |
| Glycerin | 9.1 | 7.9 |
| Outdoor Fizz Compact | 0.8 | 0.7 |
| Water | QS to 100 | QS to 100 |
| pH | 7.67 | 7.54 |

1. A personal care composition:
from about 30 to about 85 wt. % of a surfactant system comprising:
a sulfate based anionic surfactant, and
a non-sulfate based anionic surfactant;
xanthan gum; and
optionally, a fatty ester,
wherein the personal care composition has a weight ratio of sulfate based anionic surfactant to non-sulfate based anionic surfactant ranging from about 8:1 to about 1:1, and all weight percentages are based on the total weight of the personal care composition.

2. The personal care composition according to claim 1, wherein the weight ratio of the sulfate based anionic surfactant to the non-sulfate based anionic surfactant is from about 6:1 to about 2:1 or, optionally from about 5:1 to about 3:1.

3. The personal care composition according to claim 1, wherein the surfactant system is present in an amount from about 30 to about 85 wt. %; optionally, from about 35 to about 85 wt. %, from about 40 to about 85 wt. %, from about 45 to about 85 wt. %, from about 50 to about 80 wt. %, from about 50 to about 75 wt. %, or from about 50 to about 70 wt. %.

4. The personal care composition according to claim 1, wherein the sulfate based anionic sulfate comprises sodium lauryl sulfate, sodium laureth sulfate, or a combination of two or more thereof.

5. The personal care composition according to claim 1, wherein the non-sulfate based anionic surfactant is selected from acyl sulfates, acyl isethionates, acyl amino acids, acyl sarcosinates, acyl taurates, acyl glycinates, acyl glutamates, alkyl sulfonates, alkyl sulfosuccinates, alkyl sulfoacetates, alkoxylated monoacids, and a combination of two or more thereof.

6. The personal care composition according to claim 1, wherein the non-sulfate based anionic surfactant comprises sodium olefin sulfonate.

7. The personal care composition according to claim 1, wherein the sulfate based anionic surfactant is present in an amount from about 20 to about 70 wt. %; optionally, from about 30 to about 70 wt. %, from about 40 to about 70 wt. %, from about 45 to about 65 wt. %, or from about 45 to about 60 wt. %.

8. The personal care composition according to claim 1, wherein the non-sulfate based anionic surfactant is present in an amount from about 5 to about 20 wt. %; optionally, from about 7 to about 20 wt. %, from about 10 to about 20 wt. %, from about 10 to about 17, or from about 10 to about 15 wt. %.

9. The personal care composition according to claim 1, comprising a fatty ester comprising a polyglyceryl caprate.

10. The personal care composition according to claim 9, wherein the polyglyceryl caprate is selected from polyglyceryl-2-caprate, polyglyceryl-4-caprate, polyglyceryl-6-caprate, and a combination of two or more thereof.

11. A personal care composition:
a surfactant system comprising:
a sulfate based anionic surfactant, and
a non-sulfate based anionic surfactant; and
from about 0.05 to about 0.5 wt. % of xanthan gum,
wherein all weight percentages are based on the total weight of the personal care composition.

12. A diluted personal care composition comprising:
a surfactant system comprising:
a sulfate based anionic surfactant, and
a non-sulfate based anionic surfactant; and
from about 0.005 to about 0.05 wt. % of xanthan gum,
wherein the personal care composition has a weight ratio of the sulfate based anionic surfactant to the non-sulfate based anionic surfactant ranging from about 8:1 to about 1:1, and all weight percentages are based on the total weight of the personal care composition.
